(19) United States
(12) Patent Application Publication
(10) Pub. No.: US 2011/0301430 A1
(43) Pub. Date: Dec. 8, 2011

(54) VITAL SIGNS MONITOR AND HOLDER STRUCTURE

(75) Inventors: YI-HSIN HUANG, New Taipei City (TW); Ya-Ping Lin, New Taipei City (TW); Pei-Syuan Wang, New Taipei City (TW); Chih-Hui Hsieh, Zhubel City (TW); Woei-Yuh Lee, Taipei City (TW)

(73) Assignee: TAIDOC TECHNOLOGY CORPORATION

(21) Appl. No.: 13/151,544

(22) Filed: Jun. 2, 2011

(30) Foreign Application Priority Data
Jun. 4, 2010 (TW) 099118318

(51) Int. Cl. A61B 5/00

(52) U.S. Cl. 600/300

(57) ABSTRACT

A vital signs monitor and a holder structure are provided. The vital signs monitor includes a first body, a second body, a transmission line, and a detachable clamping device. The first body includes a first circuit and a first association structure. The second body includes a second circuit and a second association structure. The transmission line is electrically connected between the first circuit and the second circuit. The detachable clamping device detachable disposed between the first body and the second body for combining the first association structure and the second association structure and holding an object to locate between the first circuit and a second circuit.

### BACKGROUND OF THE INVENTION

1. Field of the Invention

The present invention generally relates to a holder structure, in particular, to a holder structure adapted to a vital signs monitor.

2. Description of Related Art

FIG. 1 and FIG. 2 are schematic views of a conventional oximeter. Referring to FIG. 1 and FIG. 2, an oximeter 100 is used to measure an oxygen concentration of the blood. The oximeter 100 includes a clamping device similar to a crocodile clip. The clamping device includes base boards 111, 112 and a spring hinge 120. A light sensor (not shown) is disposed in an inner side of the base board 111. A light source (not shown) is disposed in an inner side of the base board 112. The base board 111 and 112 respectively includes an extension part 113 and 114. A distance D1 between the base board 111, 112 can be adjusted by pressing the extension part 113 and 114, so that the finger 130 can be clamped between the light sensor and the light source. Some conventional disadvantages are listed below to show to persons skilled in the art.

1. Generally speaking, the spring hinge 120 is metal spring. The metal spring is easy to rust, and elastic fatigue. Beside, the metal spring can't be washed. If the metal spring is damaged, the user can't fix it.

2. When the light sensor and the light source are dirty, the accuracy of the oximeter may be influenced. In view of this, the user can clean the light sensor and the light source. However, the spring hinge 120 has limited angle of rotation, whereby the distance D1 is limited. When the distance D1 is not big enough, the thing that the user cleans the light sensor and the light source is not easy.

3. In the process that the oximeter 100 measures the oxygen concentration of the blood, the light sensor may be influenced by the surrounding light from the side openings 140 of the base board 111, 112. Therefore, the accuracy of the oximeter 100 may be influenced.

4. If one of the extension part 113, 114 is damaged, the distance D1 can't be adjusted, and the user can't fix it.

### SUMMARY OF THE INVENTION

Accordingly, one aspect of the present invention provides a vital signs monitor. The user can disassemble a holder structure.

Accordingly, another aspect of the present invention provides a holder structure. Whereby, convenience of a vital signs monitor may be increased.

In one aspect, the present invention provides a vital signs monitor. The vital signs monitor includes a first body, a second body, a transmission line and a detachable clamping device. The first body includes a first circuit and a first association structure. The second body includes a second circuit and a second association structure. The transmission line is electrically connected between the first circuit and the second circuit. The detachable clamping device is detachable assembled between the first body and the second body, to clamp the first association structure and the second association structure and fasten a target between the first circuit and the second circuit.

In an embodiment of the present invention, the first association structure and the second association structure correspond to a positioning structure and a positioning groove respectively.

In an embodiment of the present invention, the first association structure and the second association structure correspond to a locating post and a locating hole respectively.

In an embodiment of the present invention, the detachable clamping device is used to clamp or fasten an external surface of the first body and the second body.

In an embodiment of the present invention, the first body further includes a first fastening member and a second fastening member. The first fastening member and the second fastening member are respectively used to fasten two terminal of the detachable clamping device. The detachable clamping device is used to tie the second body.

In an embodiment of the present invention, the detachable clamping device includes a silicone rubber, a rubber, a tie, a belt or a velcro.

In an embodiment of the present invention, the vital signs monitor further includes a protection line. The protection line is connected between the first body and the second body. The protection line is shorter than the transmission line.

In an embodiment of the present invention, the detachable clamping device includes a labelable surface.

In an embodiment of the present invention, the first body further includes a first extension part. The second body further includes a second extension part. The first body and the second body contact at a fulcrum. The first extension part and the second extension part locate at one side of the fulcrum. The detachable clamping device is elastic material and disposed at another side of the fulcrum.

In an embodiment of the present invention, the vital signs monitor further includes at least one waterproofing device. The waterproofing device is disposed at a joint of the first body and/or a joint of the second body.

In another aspect, the present invention provides a vital signs monitor. The vital signs monitor includes a first body, a second body, and a detachable clamping device. The first body includes a sensing circuit and a first association structure. The second body includes a second association structure. The detachable clamping device is detachable assembled between the first body and the second body, to clamp the first association structure and the second association structure and fasten a target between the sensing circuit and the second body.

In another aspect, the present invention provides a holder structure. The holder structure includes a first housing, a second housing, and a detachable clamping device. The first housing includes a first association structure. The first housing is used to contain a sensing circuit. The second housing includes a second association structure. The detachable clamping device is detachable assembled between the first housing and the second housing, to clamp the first association structure and the second association structure and hold an object between the sensing circuit and the second body.

To sum up, the present invention utilizes a detachable clamping device to clamp a first association structure and a second association structure and fasten a target between a sensing circuit and a body. Whereby, convenience of a vital signs monitor may be increased.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1 and FIG. 2 are schematic views of a conventional oximeter.

FIG. 3 is an operating schematic view of a vital signs monitor according to a first embodiment of the present invention.

FIG. 4 is a schematic view of a vital signs monitor according to the first embodiment of the present invention.

FIG. 5 is a stretch-out view of FIG. 4.

FIG. 6 is a disarticulation schematic view of a detachable clamping device according to the first embodiment of the present invention.

FIG. 7 is an explosion diagram of a vital signs monitor according to the first embodiment of the present invention.

FIG. 8 is schematic views of a detachable clamping device according to the first embodiment of the present invention.

FIG. 9 is a schematic view of a vital signs monitor according to a second embodiment of the present invention.

FIG. 10 is a schematic view of a vital signs monitor according to a third embodiment of the present invention.

FIG. 11 is a schematic view of a vital signs monitor according to a fourth embodiment of the present invention.

FIG. 12 is a schematic view of a vital signs monitor according to a fifth embodiment of the present invention.

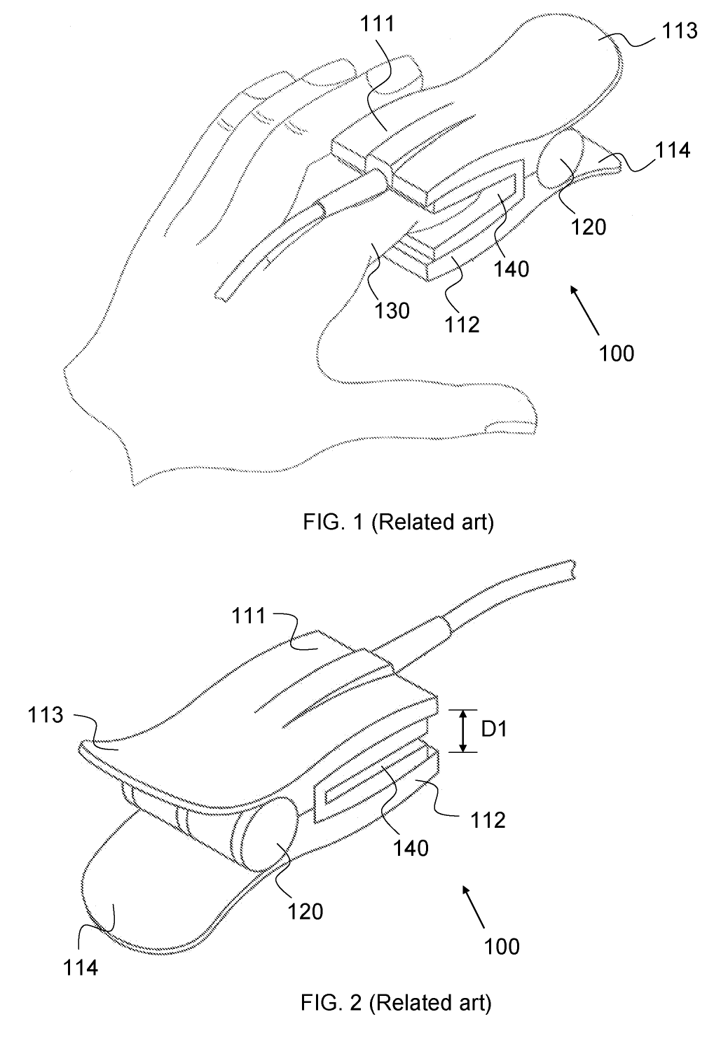

### DESCRIPTION OF THE EMBODIMENTS

Reference will now be made in detail to the present embodiments of the invention, examples of which are illustrated in the accompanying drawings. Wherever possible, the same reference numbers are used in the drawings and the description to refer to the same or like parts.

The conventional oximeter is not easy to disassemble, clean, and wash. When some element is damaged, the user can't fix it.

However, in an embodiment of the present invention, conventional spring hinge is replaced by a detachable clamping device whereby the problem that the spring hinge has limited angle of rotation may be improved, and then the vital signs monitor is easy to be cleaned. When the detachable clamping device is damaged, the user can replace it by new one. Reference will now be made in detail to the present preferred embodiments of the invention, examples of which are illustrated in the accompanying drawings. Wherever possible, the same reference numbers are used in the drawings and the description to refer to the same or like parts.

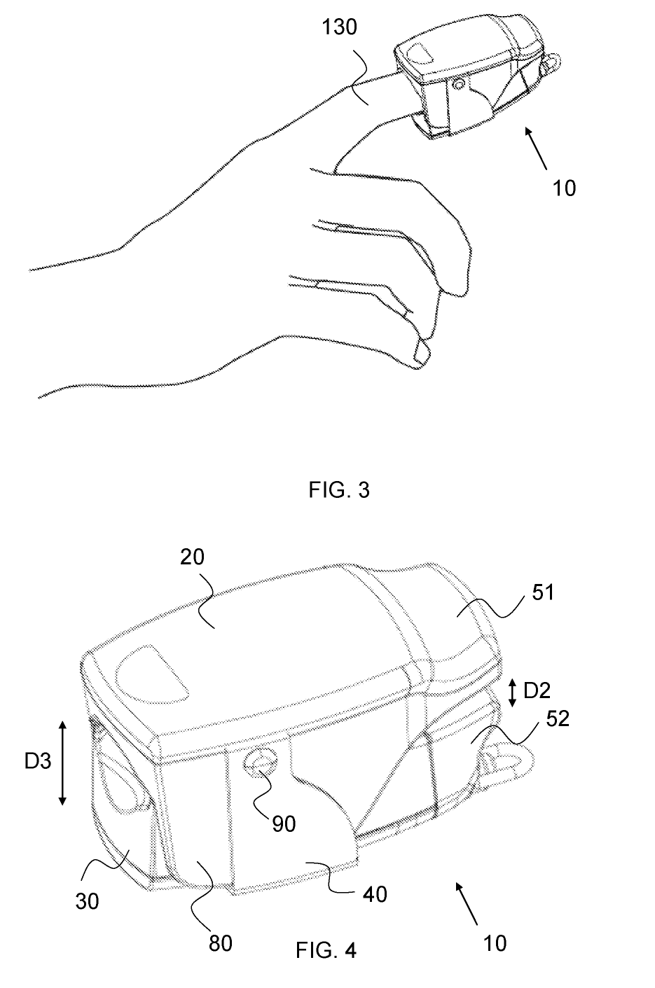

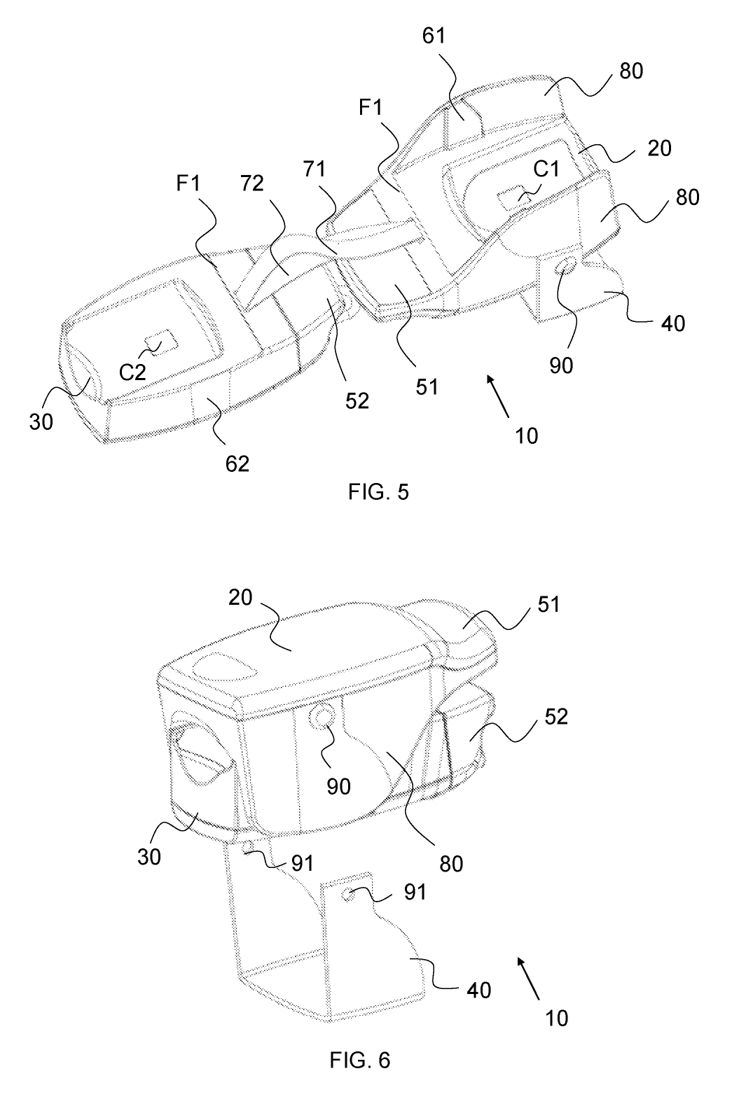

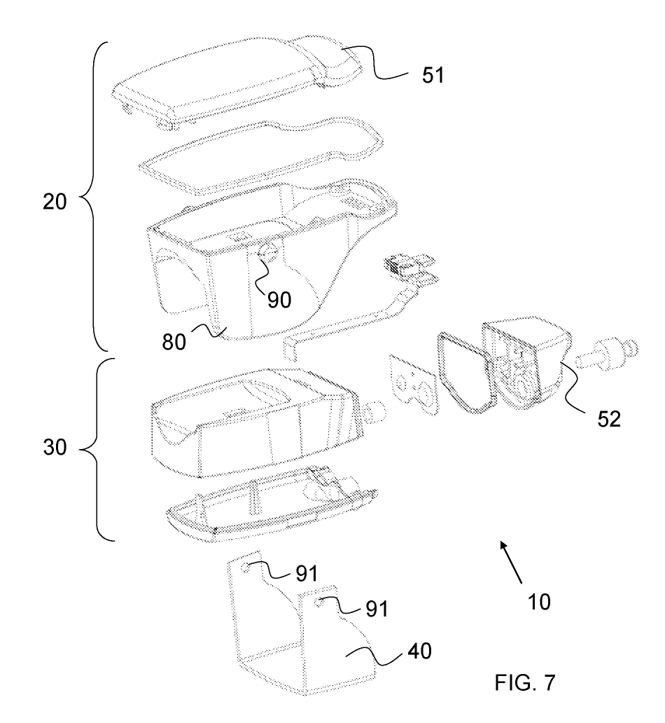

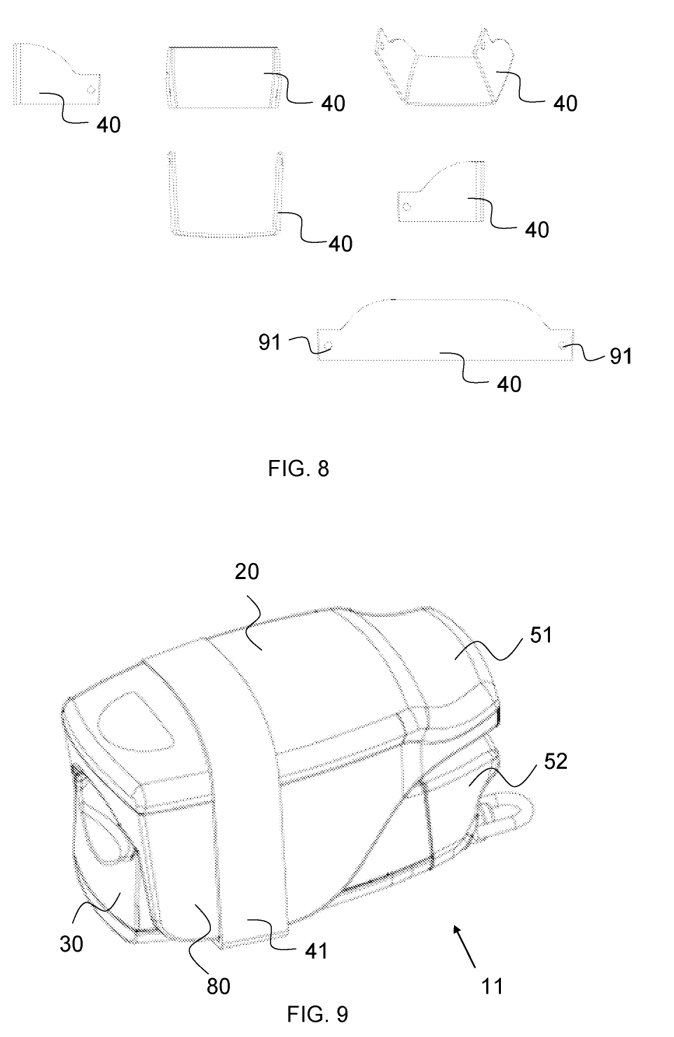

FIG. 3 is an operating schematic view of a vital signs monitor according to a first embodiment of the present invention. FIG. 4 is a schematic view of the vital signs monitor according to the first embodiment of the present invention. FIG. 5 is a stretch-out view of FIG. 4. FIG. 6 is a disarticulation schematic view of a detachable clamping device according to the first embodiment of the present invention. FIG. 7 is an explosion diagram of the vital signs monitor according to the first embodiment of the present invention. FIG. 8 is schematic views of a detachable clamping device according to the first embodiment of the present invention. Referring to FIG. 3 to FIG. 8, in the present embodiment, a vital signs monitor 10 is, for example, a transmission type oximeter, but not limited to. The vital signs monitor 10 includes a body 20, 30, a transmission line 71 and a detachable clamping device 40. The body 20 includes a housing (not shown), a circuit C1 (shown in FIG. 5) and an association structure 61. The body 30 includes a housing (not shown), a circuit C2 and an association structure 62. In the present embodiment, the association structure 61 is, for example, a protrusion part of a side wall 80, but not limited to. The association structure 62 is, for example, a concave part of side wall of the body 30, but not limited to.

The transmission line 71 is electrically connected between the circuit C1 and C2, to transmit data and control signals, and so on. The detachable clamping device 40 is detachable assembled between the body 20 and 30, to clamp the association structure 61, 62 and fasten a finger 130 between the circuit C1 and C2. The association structure 61, 62 is used to limit horizontal displacement of the body 20, 30. The circuit C1 may be disposed in a housing. The circuit C2 may be disposed in another housing. The circuit C2 includes a light source. The light source is used to emit different wavelength of light. The circuit C1 includes a light sensor. The light sensor is used to sense the light emitted by the circuit C2 and the light is passed through the finger 130. And then, the circuit C1 can analyze an oxygen concentration of the blood according to the light.

In the present embodiment, the body 20 further includes an extension part 51, a side wall 80 and two fasteners 90. Two fasteners 90 are respectively disposed in two side of the body 20, so as to fasten two terminals of the detachable clamping device 40 with the body (20). The fastener 90 is, for example, a screw nail or a rivet, and so on. The fastener 90 is, for example, composed of metal material, but not limited to. In the other embodiment, the fastener 90 may be composed of plastic material or waterproof material, and so on. In another embodiment, the fastener 90 may be integrated in the housing. For instance, use injection molding to form a hook on the housing to be served as the fastener. In addition, the detachable clamping device 40 may be composed of the elastic material and the elastic material is, for example, a silicone rubber. The detachable clamping device 40 has two openings 91. Two openings 91 are used to hook up the fasteners 90 respectively.

It should be noted, the silicone rubber has green, pollution-free, non-toxic, odorless, soft, flexible, long life, and antifouling properties, and so on. The silicone rubber is an ideal medical material to apply in medical devices. The silicone rubber may be helped to obtain the medical device certification. In addition, a surface of the silicone rubber is writable and erasable. The user or medical staff can note information on a surface of the silicone rubber to decrease property of medical errors. In another embodiment, information may be printed on the detachable clamping device 40.

In the embodiment, the body 30 further includes an extension part 52. When the vital signs monitor 10 is closed (shown in FIG. 4), the body 20 and 30 are contacted at a fulcrum F1 (shown in FIG. 5). When the vital signs monitor 10 is closed, the extension part 51 and 52 are located at one side of the fulcrum F1. The detachable clamping device 40 is disposed at another side of the fulcrum F1. When the detachable clamping device 40 is used to clamp the body 30, a force is generated to close the vital signs monitor 10.

In the other hand, the user can clamp the extension parts 51, 52 to adjust a distance D2 (shown in FIG. 4) between the extension parts 51, 52, so that a separating force is generated to close the vital signs monitor 10 to adjust an opening D3 (shown in FIG. 4) whereby the finger 130 maybe put into the opening D3 between the body 20 and 30. More specifically, the finger 130 may be disposed between the circuits C1 and C2, so as to measure an oxygen concentration of the blood. When the separating force is removed, the force generated by the detachable clamping device 40 closes the vital signs monitor 10 to fasten the finger 130 between the body 20 and 30. Therefore, the shaking of the finger 130 can be avoided to improve the accuracy.

It should be noted, the vital signs monitor 10 is easily spotted by the finger 130 so as to influence the measure result. In view of this, the user may clean the vital signs monitor 10 regularly. In the present embodiment, the detachable clamping device 40 may be unclamped from the body 30 to open the vital signs monitor 10 (as shown in FIG. 5) whereby the user can clean the inner surfaces of the body 20 and 30.

When the vital signs monitor 10 is opened, the transmission line 71 connecting between the body 20 and 30 is easy to be cut off. In view of this, a protection line 72 is disposed between the body 20 and 30 in the embodiment. The protection line 72 is not flexible and is resistant to pull. The protection line 72 is shorter than the transmission line 71 so as to protect the transmission line 71.

In the present embodiment, the detachable clamping device 40 includes an elastic material. Generally speaking, elastic material is consumable. After a long period of use, the elastic material will show flexibility in fatigue. At this point, user may disassemble the detachable clamping device 40 from the fastener 90, and then assemble a new detachable clamping device 40 on the fastener 90 whereby the problem that the detachable clamping device 40 is damaged can be solved by the user.

In the present embodiment, an appearance part of the vital signs monitor 10 includes a waterproof material. Further, waterproof materials also can be disposed in joints of the body 20 and/or the body 30, and preferably the waterproof material is, for example, a gasket or a rubber bar, and so on whereby the vital signs monitor 10 can be washed easily.

Although the above embodiment has disclosed a possible type of the vital signs monitor and the holder structure, it is common sense to the persons of ordinary skilled in this art that different manufacturers may develop different designs of the vital signs monitor and the holder structure, and the application of the present invention should not be limited to this type. In other words, as long as a detachable clamping device is utilized to fasten an object in the vital signs monitor, such implementations have conformed to the spirits of the present invention. Some other embodiments are further discussed hereinafter to allow people skilled in the art to recognize the spirit of the present invention and embody the present invention.

In the first embodiment, the vital signs monitor is used to sense the finger, but not limited to. In other embodiment, the vital signs monitor may be used to sense a different part. For example, the different part is ear lobe, tongue, lip, thenar, oral cavity, or nasal cavity, and so on.

In FIG. 4, the detachable clamping device 40 is hooked up the fastener 90, and then the detachable clamping device 40 binds the body 30 to generate a force so as to close the vital signs monitor 10, but not limited to. Persons skilled in the art modify the mounting type of the detachable clamping device 40 according to their requirement. For instance, FIG. 9 is a schematic view of a vital signs monitor according to a second embodiment of the present invention. Referring to FIG. 4 and FIG. 9, the vital signs monitor 11 is similar to the vital signs monitor 10. It should be noted, the detachable clamping device 41 of the vital signs monitor 11 is an elastic ring to bind the external surfaces of the body 20 and 30 whereby the fastener 90 in FIG. 4 can be saved. In another embodiment, grooves can be formed on the external surface of the body 20 and 30 of FIG. 9 to fit the detachable clamping device 41 whereby the displacement of the detachable clamping device 41 can be avoided.

Besides, in the first embodiment, the detachable clamping device is silicone rubber, but not limited to. In the other embodiment, the detachable clamping device may be inelastic material. More specifically, the detachable clamping device may be, for example, a rubber, a tie, a belt or a velcro, and so on.

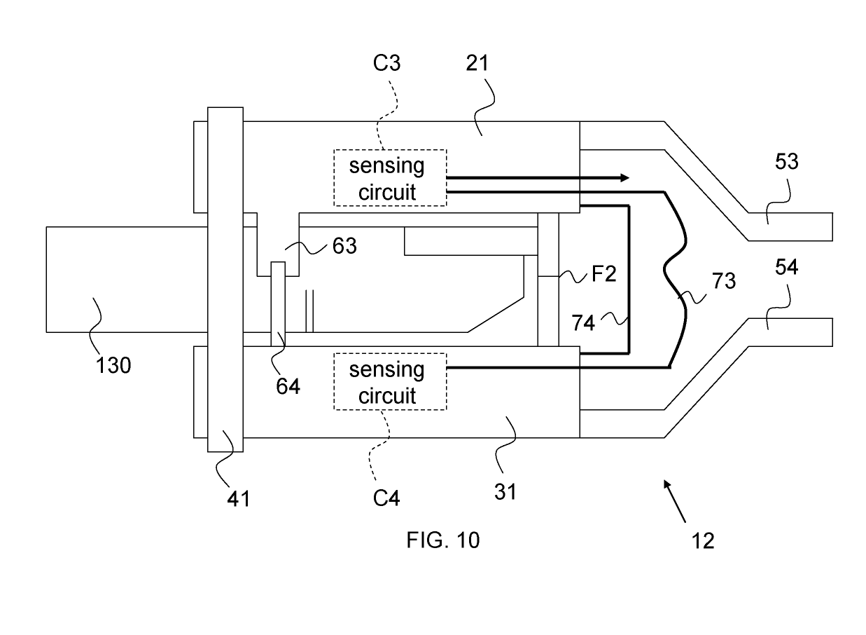

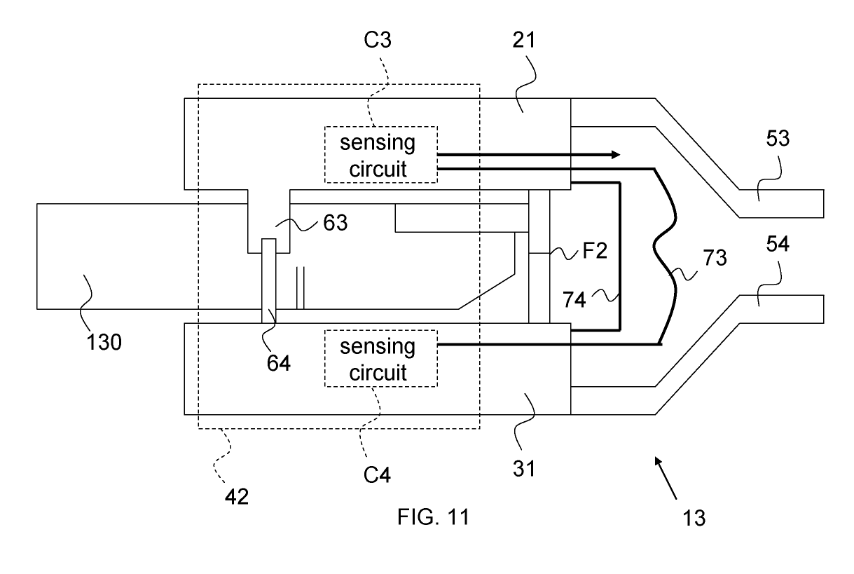

It should be known by the persons of ordinary skilled in the art that the present invention is not limited to FIG. 5. FIG. 5 is merely an optional implementation for illustration. For instance, FIG. 10 is a schematic view of a vital signs monitor according to a third embodiment of the present invention. Referring to FIG. 5 and FIG. 10, a vital signs monitor 12 is similar to the vital signs monitor 10. It should be noted that the extension parts 51 and 52 of FIG. 5 are replaced by extension parts 53 and 54 of FIG. 10, the fulcrum F1 of FIG. 5 is replaced by a fulcrum F2 of FIG. 10, the positioning structures 61 and 62 of FIG. 5 are replaced by a locating post 64 and a locating hole 63 of FIG. 10, and the transmission line 71 and the protection line 72 are replaced by a transmission line 73 and a protection line 74 of FIG. 10. Moreover, in FIG. 10, a body 21 includes a sensing circuit C3, the sensing circuit C3 includes a light source, a body 31 includes a sensing circuit C4, and the sensing circuit C4 includes a light sensor whereby functions of the vital signs monitor 12 are similar to that of the vital signs monitor 10.

A width of the detachable clamping device 40 of FIG. 10 is an optional implementation for illustration. The persons of ordinary skilled in the art may adjust the width of the detachable clamping device 40. For example, FIG. 11 is a schematic view of a vital signs monitor according to a fourth embodiment of the present invention. In the present embodiment, a detachable clamping device 42 of a vital signs monitor 13 not only provides a force to clamp the body 21 and 31, but also has a side wall function to decrease interference of ambient light. Furthermore, the detachable clamping device 42 has a protection function. The detachable clamping device 42 covers the external surface of the body 21 and 31 to achieve the function of shock-proof. The detachable clamping device 42 includes a buffer material. The buffer material is, for example, a silicone rubber, a rubber, a fabric, or a textile, and so on.

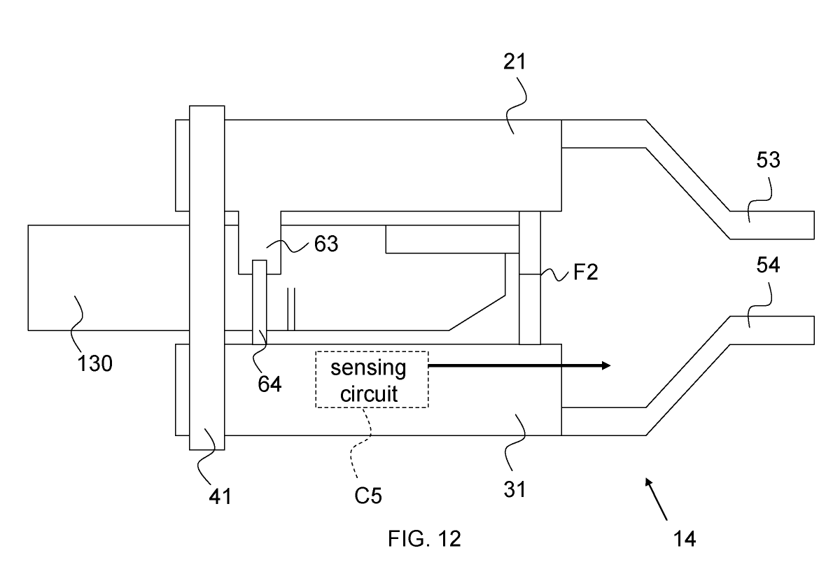

In the first embodiment, the vital signs monitor is a transmission-typed oximeter, but not limited to. For instance, FIG. 12 is a schematic view of a vital signs monitor according to a fifth embodiment of the present invention. Referring to FIG. 12, in the present embodiment, a vital signs monitor 14 is reflection-typed oximeter. A sensing circuit C5 is disposed in a body 31, wherein the sensing circuit C5 includes a light sensor and a light source whereby not only the vital signs monitor 14 can be used to measure an oxygen concentration of the blood, but also a transmission line and a protection line can be saved.

The vital signs monitor of the first embodiment uses the optical detection technique, but not limited to. In other embodiments, the vital signs monitor may use the wireless signal detection technique. The wireless signal detection technique is, for example, a radio frequency detection technique, a magnetic detection technique, a voltage detection technique, or a current detection technique, and so on. Further, the vital signs monitor in the present invention is not limited to measure an oxygen concentration of the blood. For example, the vital signs monitor may have different functions, if different sensing circuits are disposed in the vital signs monitor. More specifically, the vital signs monitor may be used to measure the vital signs of blood flow, blood volume, blood glucose concentration, heart rate, or body fat . . . etc.

To sum up, the detachable clamping device of the vital signs monitor is utilized to fasten a target between two bodies whereby convenience of the vital signs monitor may be increased. Further, embodiments of the present invention also have the following advantages.

1. Association structures are disposed in the two bodies respectively to help combining the bodies. Further, the horizontal displacement of the bodies can be avoided.

2. The detachable clamping device may be served as a side wall to decrease inference of surrounding light. Further, the detachable clamping device may be served as a protection cover to protect the vital signs monitor.

3. The silicone rubber may be helped to obtain the medical device certification.

4. The user or medical staff can note information on the labelable surface of the detachable clamping device to decrease property of medical errors.

5. The conventional crocodile clip is replaced by the detachable clamping device and the extension part, and the detachable clamping device includes elastic material.

6. The protection line can be used to protect the transmission line.

7. The vital signs monitor has waterproof function preferably, and then the vital signs monitor can be washed, and the life of the vital signs monitor can be extended.

Though the present invention has been disclosed above by the preferred embodiments, they are not intended to limit the present invention. Anybody skilled in the art can make some modifications and variations without departing from the spirit and scope of the present invention. Therefore, the protecting range of the present invention falls in the appended claims and their equivalents.

What is claimed is:

1. A vital signs monitor, comprising:
a first body, including a first circuit and a first association structure;
a second body, including a second circuit and a second association structure;
a transmission line, electrically connecting between the first circuit and the second circuit; and
a detachable clamping device, detachable assembling between the first body and the second body, to clamp the first association structure and the second association structure and fasten a target between the first circuit and the second circuit.

2. The vital signs monitor according to claim 1, wherein the first association structure and the second association structure are respectively a positioning structure and a corresponding positioning groove.

3. The vital signs monitor according to claim 1, wherein the first association structure and the second association structure are respectively a locating post and a corresponding locating hole.

4. The vital signs monitor according to claim 1, wherein the detachable clamping device is used to clamp or fasten an external surface of the first body and the second body.

5. The vital signs monitor according to claim 1, wherein the first body further includes a first fastening member and a second fastening member, the first fastening member and the second fastening member are respectively used to fasten two terminals of the detachable clamping device, and the detachable clamping device is used to tie the second body.

6. The vital signs monitor according to claim 1, wherein the detachable clamping device includes a silicone rubber, a rubber, a tie, a belt or a velcro.

7. The vital signs monitor according to claim 1, further comprising:
a protection line, connecting between the first body and the second body, wherein the protection line is shorter than the transmission line.

8. The vital signs monitor according to claim 1, wherein the detachable clamping device includes a labelable surface.

9. The vital signs monitor according to claim 1, wherein the first body further includes a first extension part, the second body further includes a second extension part, the first body and the second body contact at a fulcrum, the first extension part and the second extension part locate at one side of the fulcrum, the detachable clamping device is elastic material and disposed at another side of the fulcrum.

10. The vital signs monitor according to claim 1, further comprising:
at least one waterproofing device, disposing at a joint of the first body or a joint of the second body.

11. A vital signs monitor, comprising:
a first body, including a sensing circuit and a first association structure;
a second body, including a second association structure; and
a detachable clamping device, detachable assembling between the first body and the second body, to clamp the first association structure and the second association structure and fasten a target between the sensing circuit and the second body.

12. A holder structure, comprising:
a first housing, including a first association structure, wherein the first housing is used to contain a sensing circuit;
a second housing, including a second association structure; and
a detachable clamping device, detachable assembling between the first housing and the second housing, to clamp the first association structure and the second association structure and hold an object between the sensing circuit and the second body.
